(19) United States
(12) Patent Application Publication
(10) Pub. No.: US 2025/0188548 A1
(43) Pub. Date: Jun. 12, 2025

(54) METHOD FOR EARLY DETECTION, PREDICTION OF TREATMENT RESPONSE AND PROGNOSIS OF LUNG CANCER

(71) Applicant: EG BIOMED CO., LTD., Taipei City (TW)

(72) Inventors: Ruo-Kai LIN, Taipei City (TW); Hsieh-Tsung SHEN, Taipei City (TW)

(21) Appl. No.: 18/977,532

(22) Filed: Dec. 11, 2024

Related U.S. Application Data
(60) Provisional application No. 63/608,668, filed on Dec. 11, 2023.

(51) Int. Cl. C12Q 1/6886; C12Q 1/6851

(52) U.S. Cl. CPC C12Q 1/6886; C12Q 1/6851; C12Q 2600/118; C12Q 2600/154

(57) ABSTRACT

The present disclosure discloses a set of novel epigenetic biomarkers for early detection, prediction of treatment response and prognosis of lung cancer. Aberrant methylation of the epigenetic biomarkers can be detected in tumor tissues and plasma samples from lung cancer patients but not in normal individuals. The present disclosure also discloses primers and kits used herein.

### PRIORITY INFORMATION

This application claims benefit of and priority to U.S. Provisional Patent Application No. 63/608,668, filed Dec. 11, 2023, the contents of which is incorporated by reference in its entirety.

### SEQUENCE LISTING

The instant application contains a Sequence Listing which has been filed electronically in XML format and is hereby incorporated by reference in its entirety. Said XLM copy, created on 10 Dec. 2024, is named G4590-18800US_20241210_SeqListing.xml and is 9,115 bytes in size.

### FIELD OF THE INVENTION

The present disclosure relates to epigenetic biomarkers for prediction of risk or susceptibility of lung cancer. Particularly, the disclosure provides a method for early detection, prediction of treatment response and prognosis of lung cancer based on methylation statuses of gene biomarkers.

### BACKGROUND OF THE INVENTION

Cancer is a group of diseases involving abnormal cell growth with the potential to invade or spread to other parts of the body and is a leading cause of deaths worldwide.

Methylated DNA has been studied as a potential class of biomarkers in the tissues of most tumor types. In many instances, DNA methyltransferases add a methyl group to DNA at cytosine-phosphate-guanine (CpG) island sites as an epigenetic control of gene expression.

However, the current techniques in detection of lung cancer are not satisfied.

### SUMMARY OF THE INVENTION

The present disclosure discloses one or more novel epigenetic biomarkers for early detection, prediction of treatment response and prognosis of lung cancer. Aberrant methylation of the epigenetic biomarkers is detected in tumor tissues and plasma samples from cancer patients but not in normal individuals. The present disclosure also discloses primers used herein.

In one embodiment, the present disclosure provides a method for assaying a methylation level in a human subject who is in a need of detecting a predisposition to lung cancer or predicting likelihood, treatment response, prognosis or recurrence of the lung cancer, comprising assaying the methylation level of a target DNA sequence in one or more target genes selected from CG-15, LINC00682 and NBPF13P, wherein the presence of hypermethylation in the target DNA sequence of the subject is indicative of the predisposition to, likelihood, poor treatment response, poor prognosis or recurrence of lung cancer.

In some embodiments, the method as disclosed herein further comprises steps of
providing a biological sample containing one or more target DNA sequences from the human subject and extracting the DNA sequences from the biological sample;
treating the extracted DNA with bisulfate;
amplifying the bisulfite-treated DNA with a primer pair specific to the target DNA sequence; and
assaying the methylation level of one or more CpG sites in the target DNA sequence by a polymerase chain reaction.

In one embodiment, the method as disclosed herein further comprises steps of:
providing (i) a biological sample containing DNA from the human subject and extracting cell-free DNA from the biological sample or (ii) a biological sample from a lung tumor tissue of the human subject and extracting the genomic DNA from the biological sample;
treating the extracted cell-free DNA or the extracted genomic DNA with bisulfate;
amplifying the bisulfite-treated DNA with at least one of primer pairs 1 to 3; and
measuring the methylation level of one or more CpG sites in target DNA sequence by a polymerase chain reaction.

In some embodiments, wherein the cell-free DNA or the genomic DNA comprises the target DNA sequence in the one or more target genes.

In one embodiment, the present disclosure provides a method for assaying a methylation level in a biological sample from a human subject who is in a need of detecting a predisposition to lung cancer or predicting likelihood, treatment response, prognosis or recurrence of the lung cancer, comprising
(a) providing a biological sample containing a target DNA sequence in one or more target genes selected from CG-15, LINC00682 and NBPF13P from the human subject, and
(b) assaying the methylation level of the target DNA sequence in CG2-15, wherein the target DNA sequence comprises a fragment amplified by a primer pair 1 having the nucleotide sequence of SEQ ID NOs: 1 (GTT AGT CGT GGG GTA AGG AGG TC) and 2 (CCG AAA AAA ATA AAC TAA ACC CGA A) or fragment thereof having an identity of about 85%, about 86%, about 87%, about 88%, about 89%, about 90%, about 91%, about 92%, about 93%, about 94%, about 95%, about 96%, about 97%, about 98%, about 99%, or higher percent to the primer pair 1;
assaying the methylation level of the target DNA sequence in LINC00682, wherein the target DNA sequence comprises a fragment amplified by a primer pair 2 having the nucleotide sequence of SEQ ID NOs: 3 (AGT TTA GGA TCG GTA GGG TTT TCG T) and 4 (ATT CGA CAC CTA TCC AAC TAC TCC G) or a fragment thereof having an identity of about 85%, about 86%, about 87%, about 88%, about 89%, about 90%, about 91%, about 92%, about 93%, about 94%, about 95%, about 96%, about 97%, about 98%, about 99%, or higher percent to the primer pair 2, and/or
assaying the methylation level of the target DNA sequence in NBPF13P, wherein the target DNA sequence comprises a fragment amplified by a primer pair 3 having the nucleotide sequence of SEQ ID NOs: 5 (AGC GTT TAT AGA GTG TGG AAT CGT C) and 6 (TTT CTC CTA CTA ATA TCT ACC CGC C) or a fragment thereof having an identity of about 85%, about 86%, about 87%, about 88%, about 89%, about 90%, about 91%, about 92%, about 93%, about 94%, about 95%, about 96%, about 97%, about 98%, about 99%, or higher percent to the primer pair 3,
wherein the presence of hypermethylation in the target DNA sequence of the human subject is indicative of the predisposition to, likelihood, poor treatment response, poor prognosis or recurrence of lung cancer.

In some embodiments, a target DNA sequence methylation specific primer is used to assay the methylation level of the target DNA sequence and the control DNA sequence in the biological sample.

Examples of the specific primers include, but are not limited to, primer pairs 1 to 3 and a fragment thereof having an identity of about 85%, about 86%, about 87%, about 88%, about 89%, about 90%, about 91%, about 92%, about 93%, about 94%, about 95%, about 96%, about 97%, about 98%, about 99%, or higher percent to any of the above primers.

In some embodiments, the lung cancer described herein is squamous cell carcinoma or adenocarcinoma.

In one embodiment, the presence of hypermethylation in the target DNA sequence of the human subject is determined by comparing the methylation level of the target DNA sequence to a methylation level of a control DNA sequence. In some embodiments, the present disclosure provides a method for detecting a predisposition to lung cancer or predicting likelihood, treatment response, prognosis or recurrence of lung cancer in a subject, comprising (a) providing a biological sample from a human subject comprising a target DNA sequence comprising the fragments amplified by the primer pairs 1 to 3 or the fragment, and (b) assaying methylation levels of the target DNA sequence and of a control DNA sequence in the biological sample using a target DNA sequence methylation specific probe or a target DNA sequence methylation specific primer, (c) measuring a relative methylation level of the target DNA sequence compared to a control DNA sequence, (d) identifying the human subject as having the predisposition to, likelihood, poor treatment response, poor prognosis or recurrence of, lung cancer when hypermethylation is present in the relative methylation level. In a further embodiment, the control DNA sequence is in a normal tissue as a control sample without lung cancer.

In some embodiments, the biological sample described herein is a tissue, cell, blood, urine, serum, plasma, stool, ascites, sputum, saliva, gastric juice, bile, or oral mucosa.

In some embodiments, the methylation level is measured by a polymerase chain reaction (such as methylation-specific PCR (MSP), quantitative methylation-specific PCR (QMSP), methylation DNA immunoprecipitation PCR (MeDIP)), nucleic acid sequencing (such as bisulfite sequencing or pyrosequencing), bisulfite conversion, mass spectrometry, methylation specific nuclease, mass-based separation, target capture or microarray. In a particular embodiment, the methylation level is detected by a polymerase chain reaction.

In some embodiments, the method described herein is for measuring a methylation level in a human subject who has a need of detection of lung cancer.

Certain embodiments of the target DNA sequence described herein includes any of the following combinations of the DNA sequences:
the fragment amplified by the primer pair 1 or the fragment thereof and the fragment amplified by the primer pair 2 or the fragment thereof,
the fragment amplified by the primer pair 1 or the fragment thereof and the fragment amplified by the primer pair 3 or the fragment thereof,
the fragment amplified by the primer pair 2 or the fragment thereof and the fragment amplified by the primer pair 3 or the fragment thereof, and
the fragment amplified by the primer pair 1 or the fragment thereof, the fragment amplified by the primer pair 2 or the fragment thereof and the fragment amplified by the primer pair 3 or the fragment thereof.

In a further embodiment, measuring the methylation level described herein further comprises a step of measuring the specificity and sensitivity by a weighted sum score analysis.

In a further embodiment, the method described herein further comprises a step of administering an anti-lung cancer agent to the subject.

In another aspect, the present disclosure provides a method for assaying a methylation level in a biological sample from a human subject who is in a need of detecting a predisposition to lung cancer or predicting likelihood, treatment response, prognosis or recurrence of the lung cancer, comprising
providing a biological sample containing a target DNA sequence in one or more target genes selected from CG-15, LINC00682 and NBPF13P from the human subject;
assaying methylation levels of the CG-15, LINC00682 and NBPF13P from the DNA sample using methylation specific polymerase chain reaction assay (MSP), wherein the probes used in the assay comprises SEQ ID NO: 7 for CG-15, SEQ ID NO: 8 from LINC00682 and SEQ ID NO: 9 for NBPF13P;
comparing the methylation levels of the previous step to methylation levels from a control sample without lung cancer; and
identifying the subject as having the predisposition to, likelihood, poor treatment response, poor prognosis or recurrence of, lung cancer when hypermethylation is present in the relative methylation level.

In some embodiments, the following forward and reverse primers are further used in the assay: a primer pair 1 having the amino acid sequence of SEQ ID NOs: 1 and 2 or a fragment thereof having an identity of about 85%, about 86%, about 87%, about 88%, about 89%, about 90%, about 91%, about 92%, about 93%, about 94%, about 95%, about 96%, about 97%, about 98%, about 99%, or higher percent, a primer pair 2 having the amino acid sequence of SEQ ID NOs: 3 and 4 or a fragment thereof having an identity of about 85%, about 86%, about 87%, about 88%, about 89%, about 90%, about 91%, about 92%, about 93%, about 94%, about 95%, about 96%, about 97%, about 98%, about 99%, or higher percent, and a primer pair 3 having the amino acid sequence of SEQ ID NOs: 5 and 6 or a fragment thereof having an identity of about 85%, about 86%, about 87%, about 88%, about 89%, about 90%, about 91%, about 92%, about 93%, about 94%, about 95%, about 96%, about 97%, about 98%, about 99%, or higher percent.

In one embodiment, the method described herein can be used in early predication of a lung cancer.

In some embodiments, the one or more target genes described herein are CG2-15; LINC00682; NBPF13P; CG2-15 and LINC00682; CG2-15 and NBPF13P; LINC00682 and NBPF13P; or CG-15, LINC00682 and NBPF13P.

The present disclosure provides an isolated nucleic acid molecule having a sequence selected from the group consisting of:
a primer pair 1 having the amino acid sequence of SEQ ID NOs: 1 and 2 or a fragment thereof having an identity of about 85%, about 86%, about 87%, about 88%, about 89%, about 90%, about 91%, about 92%, about 93%, about 94%, about 95%, about 96%, about 97%, about 98%, about 99%, or higher percent, a primer pair 2 having the amino acid sequence of SEQ ID NOs: 3 and 4 or a fragment thereof having an identity of about 85%, about 86%, about 87%, about 88%, about 89%, about 90%, about 91%, about 92%, about 93%, about 94%, about 95%, about 96%, about 97%, about 98%, about 99%, or higher percent, and a primer pair 3 having the amino acid sequence of SEQ ID NOs: 5 and 6 or a fragment thereof having an identity of about 85%, about 86%, about 87%, about 88%, about 89%, about 90%, about 91%, about 92%, about 93%, about 94%, about 95%, about 96%, about 97%, about 98%, about 99%, or higher percent.

In some embodiments, the sample providing step described herein is to provide (i) a biological sample containing DNA from the human subject and extracting cell-free DNA from the sample or (ii) a biological sample from a lung tumor tissue of the human subject and extracting the genomic DNA from the sample, wherein the cell-free DNA or the genomic DNA comprises the target DNA sequence in the one or more target genes.

The present disclosure also provides a kit for detecting a predisposition to lung cancer or predicting likelihood, treatment response, prognosis or recurrence of lung cancer in a subject, which comprises an isolated nucleic acid molecule for assaying a methylation level of the target DNA sequence as described herein. The kit may further comprise sodium bisulfite and adapters for whole target genes amplification, and polynucleotides (e.g., detectably-labeled polynucleotides) to quantify the presence of a methylated and/or an unmethylated cytosine residue in the target DNA sequence in one or more target genes as described herein. Furthermore, the kit may further comprise a methylation sensing restriction enzyme for whole target sequence or genes amplification.

### BRIEF DESCRIPTION OF THE DRAWINGS

FIG. 1 shows the difference of methylation level (β value) of target nucleic acid and genes between lung adenocarcinoma tissues and adjacent normal tissues. Darker color indicates tissues with higher methylation level according to Illumina™ Methylation 450K array-based data. 1: cg06936155; 2: cg03974889; 3: cg07995570; 4: cg16005939; 5: cg01984304; 6: cg02368058; 7: cg 13456504; 8: cg22167515; 9: cg24347994; 10: cg 14663264; 11: cg20041088; 12: cg13844193; 13: cg14592399; 14: cg15553500; 15: cg16258337; 16: cg24947764; 17: cg01122241; 18:01370541; 19: cg02881929; 20: cg03905413; 21: cg04384626; 22: cg17231999; 23:18351939; 24: cg20038996; 25:24722073; 26: cg27405791; 27: cg10124710; 28: cg16237891; 29: cg17856063; 30: cg23019889; 31: cg03526459; 32: cg06317351; 33: cg11655389; 34: cg22671717; 35: cg02487331; 36: cg03229033; 37: cg20449590; 38: cg06871074; 39: cg21908235; 40: cg00044796.

FIG. 2 shows the difference of methylation level (β value) of target nucleic acid and genes between lung squamous cell carcinoma and adjacent normal tissues. Darker color indicates tissues with higher methylation level according to Illumina™ Methylation 450K array-based data. 1: cg06936155; 2: cg03974889; 3: cg07995570; 4: cg16005939; 5: cg01984304; 6: cg02368058; 7: cg 13456504; 8: cg22167515; 9: cg24347994; 10: cg 14663264; 11: cg20041088; 12: cg13844193; 13: cg14592399; 14: cg15553500; 15: cg16258337; 16: cg24947764; 17: cg01122241; 18:01370541; 19: cg02881929; 20: cg03905413; 21: cg04384626; 22: cg17231999; 23:18351939; 24: cg20038996; 25:24722073; 26: cg27405791; 27: cg10124710; 28: cg16237891; 29: cg17856063; 30: cg23019889; 31: cg03526459; 32: cg06317351; 33: cg11655389; 34: cg22671717; 35: cg02487331; 36: cg03229033; 37: cg20449590; 38: cg06871074; 39: cg21908235; 40: cg00044796.

FIG. 3 shows the difference in early detection of the methylation level of epigenetic biomarkers of target genes in plasma samples of healthy subjects and lung cancer patients.

FIG. 4 shows the differences in early detection of the methylation level of target genes in plasma samples of healthy subjects and lung cancer patients in pilot study.

FIG. 5 shows the differences in early detection of the methylation level of target genes in plasma samples from the expanded cohort.

FIG. 6 shows the clinical performance based on the methylation level of target genes in plasma samples.

FIG. 7 shows a comparative analysis of the clinical performance between the methylation levels of NBPF13P and CG2-15 versus CEA in identifying lung cancer probability.

FIG. 8 shows that the target DNA sequences and methylation level are used in predicting treatment response of lung cancer.

### DETAILED DESCRIPTION OF THE INVENTION

It is understood that this invention is not limited to the particular materials and methods described herein. It is also to be understood that the terminology used herein is for the purpose of describing particular embodiments and is not intended to limit the scope of the present invention, which will be limited only by the appended claims.

It must be noted that, as used in this specification and the appended claims, the singular forms “a,” “an,” and “the” include plural referents unless the content clearly dictates otherwise. Thus, for example, reference to “a biomarker” includes a mixture of two or more biomarkers, and the like.

The term “AUC” as used herein is an abbreviation for the area under a curve. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better, optimum is 1, a random test would have a ROC curve lying on the diagonal with an area of 0.5; for reference: J. P. Egan. Signal Detection Theory and ROC Analysis, Academic Press, New York, 1975).

The term “a biological sample” refers to a sample of tissue, cells, or fluid isolated from a subject, including but not limited to, for example, blood, buffy coat, plasma, serum, blood cells (e.g., peripheral blood mononucleated cells (PBMCS), band cells, neutrophils, metamyelocytes, monocytes, or T cells), fecal matter, urine, bone marrow, bile, stool, ascites, sputum, spinal fluid, lymph fluid, samples of the skin, external secretions of the skin, respiratory, intestinal, and genitourinary tracts, tears, saliva, milk, organs, biopsies and also samples of in vitro cell culture constituents, including, but not limited to, conditioned media resulting from the growth of cells and tissues in culture medium, e.g., recombinant cells, and cell components.

The term “a biomarker” refers to a nucleic acid molecule which is present in a sample taken from patients having human cancer as compared to a comparable sample taken from control subjects (e.g., a person with a negative diagnosis or undetectable cancer, normal or healthy subject). The biomarker can be a nucleic acid, a fragment of a nucleic acid, a polynucleotide, or an oligonucleotide that can be detected and/or quantified. Biomarkers include polynucleotides comprising nucleotide sequences from genes.

The term “a CpG island” as used herein refers to stretches of DNA in a genome that are rich in GC relative to the rest of the genome. Typically, the GC content is 50% or greater in these regions, which extend over hundreds of base pairs and sometimes thousands. Often these regions mark the 5′ ends of genes.

As used herein, the term “early detection” of cancer refers to discovering the likelihood of cancer before metastasis. Preferably, it refers to discovering the likelihood of cancer before a morphological change in a sample tissue or cell is observed.

As used herein, the terms “detect”, “detecting” or “detection” may describe either the general act of discovering or discerning or the specific observation of a detectably labeled composition.

As used herein, the term “homology” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In some embodiments, the degree of homology between the two sequences is sufficient to allow homologous recombination therebetween, under appropriate stringent conditions.

In some embodiments, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. See, e.g., Sambrook et al., supra; Nucleic Acid Hybridization: A Practical Approach, editors B. D. Hames and S. J. Higgins, (1985) Oxford; Washington, D.C.; IRL Press).

As used herein, the term “prediction” refers to the likelihood that a patient will respond either favorably or unfavorably to a drug or set of drugs, and also the extent of those responses. Thus, treatment predictive factors are variables related to the response of an individual patient to a specific treatment, independent of prognosis.

The term “methylation,” as used herein, refers to the presence of a methyl group added by the action of a DNA methyl transferase enzyme to a cytosine base or bases in a region of nucleic acid, e.g., genomic DNA.

The term “methylation level” of a nucleic acid molecule refers to the presence or absence of one or more methylated nucleotide bases in the nucleic acid molecule. For example, a nucleic acid molecule containing a methylated cytosine is considered methylated (i.e., the methylation state of the nucleic acid molecule is methylated). A nucleic acid molecule that does not contain any methylated nucleotides is considered unmethylated.

The term “hypermethylation” refers to the average methylation state corresponding to an increased presence of methylated nucleotide bases in the nucleic acid molecule at one or a plurality of CpG dinucleotides within a DNA sequence of a test DNA sample, relative to the amount of methylated nucleotide bases in the nucleic acid molecule found at corresponding CpG dinucleotides within a normal control DNA sample.

The term “subject” refers to humans.

The term “susceptibility” refers to a constitution or condition of the body which makes the tissues react in special ways to certain extrinsic stimuli and thus tends to make the individual more than usually susceptible to certain diseases.

The term “target site” or “target sequence” refers to a nucleic acid sequence that defines a portion of a nucleic acid to which a binding molecule will bind, provided sufficient conditions for binding exist.

The term “risk” refers to the estimated chance of getting a disease during a certain time period, such as within the next 10 years, or during the subject's lifetime.

The term “prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease.

The term “weight sum score” refers to every possible alternative being rated by a score including all objectives, individually weighted to stress the importance of different objectives.

As used herein, the term “nucleic acid molecule” (or “nucleic acid” or “polynucleotide”) may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide”. A nucleic acid molecule is usually at least 10 bases in length, unless otherwise specified. The term may refer to a molecule of RNA or DNA of indeterminate length. The term includes single- and double-stranded forms of DNA. A nucleic acid molecule may include either or both naturally-occurring and modified nucleotides linked together by naturally occurring and/or non-naturally occurring nucleotide linkages.

Cancer is characterized by an abnormal growth of a cell caused by one or more mutations or modifications of a gene leading to dysregulated balance of cell proliferation and cell death. In many disease processes, such as cancer, gene promoter CpG islands acquire abnormal hypermethylation, which results in transcriptional silencing that can be inherited by daughter cells following cell division. DNA methylation causing silencing in cancer typically occurs at multiple CpG sites in the CpG islands that are present in the promoters of protein coding genes. Alterations of DNA methylation have been recognized as an important component of cancer development. DNA methylation profiling provides higher clinical sensitivity and dynamic range compared to other cancer detections. Accordingly, the present disclosure provides a method and kit for early prediction, treatment response and prognosis or recurrence monitoring of lung cancer.

In the present disclosure, the methylation level of target DNA sequences or a fragment thereof in a biological sample are measured to detect lung cancer or detect a predisposition to lung cancer or predict treatment response, prognosis or recurrence of lung cancer in a human subject.

In some embodiments, the methylation comprises a cytosine methylation site. In some instances, cytosine methylation comprises 5-methylcytosine (5-mCyt) and 5-hydroxymethylcytosine. In some cases, a cytosine methylation site occurs in a CpG dinucleotide motif. In other cases, a cytosine methylation site occurs in a CHG or CHH motif, in which is adenine, cytosine or thymine. In some instances, one or more CpG dinucleotide motif or CpG site forms a CpG island, a short DNA sequence rich in CpG dinucleotide. In some instances, CpG islands are typically, but not always, between about 0.2 to about 1 kb in length. In some instances, the methylation comprises CpG island methylation.

In some embodiments, the treatment of biological sample includes providing (i) a biological sample containing DNA from the human subject and extracting cell-free DNA from the sample or (ii) a biological sample from a lung tumor tissue of the human subject and extracting the genomic DNA from the sample. The cell-free DNA or the genomic DNA comprises the target DNA sequence(s) in the target DNA sequence(s). The methylation status is analyzed by a methylation specific enzymatic digest; bisulfite sequencing; an analysis selected from promoter methylation, CpG island methylation, MSP, HeavyMethyl, MethyLight, and Ms-SNuPE; and other methods relying on a detection of amplified DNA. The term “MethyLight™” refers to a fluorescence-based real-time PCR technique. MethylLight is described by Eads et al., Cancer Res. 59:2302-2306, 1999, herein incorporated by reference.

The term “HeavyMethyl” assay, refers to an assay wherein methylation specific blocking probes (also referred to herein as blockers) covering CpG positions between, or covered by the amplification primers enable methylation-specific selective amplification of a nucleic acid sample.

The term “Ms-SnuPE” refers to Methylation-sensitive Single Nucleotide Primer Extension. MsSNuPE is described by Gonzalgo & Jones, Nucleic Acids Res. 25:2529-2531, 1997, herein incorporated by reference.

The term “MSP” refers to Methylation-specific PCR. MSP is described by Herman et al. Proc. Natl. Acad. Sci. USA 93:9821-9826, 1996, and by U.S. Pat. No. 5,786,146, each of which are herein incorporated by reference.

Bisulfite modification of DNA is a method to assess CpG methylation level. 5-methylcytosine is the most frequent covalent base modification in the DNA of eukaryotic cells. However, 5-methylcytosine positions cannot be identified directly by sequencing or hybridization methods, because 5-methylcytosine has the same base pairing behavior as cytosine. Moreover, the epigenetic information carried by 5-methylcytosine is completely lost during, e.g., PCR amplification. Bisulfite sequencing is a method for analyzing DNA for the presence of 5-methylcytosine is based upon the specific reaction of bisulfite with cytosine whereby, upon subsequent alkaline hydrolysis, cytosine is converted to uracil which corresponds to thymine in its base pairing behavior. However, 5-methylcytosine remains unmodified under the aforementioned conditions. Thus, the original DNA is converted in such a manner that methylcytosine, which originally could not be distinguished from cytosine by its hybridization behavior, can be detected as the only remaining cytosine using molecular biological techniques, for example, by amplification and hybridization, or by sequencing.

In one embodiment, the methylation level is detected by polymerase chain reaction, nucleic acid sequencing (such as bisulfite sequencing or pyrosequencing), bisulfite conversion, mass spectrometry, methylation specific nuclease, mass-based separation, target capture or microarray. In one embodiment, the methylation level is detected by using primers to amplify a methylated CpG of the target genes. In a further embodiment, the detection of methylation is conducted by PCR, methylation specific PCR (MSP), real-time methylation specific PCR, quantitative methylation-specific PCR (QMSP), PCR using a methylated DNA-specific binding protein or quantitative PCR.

In one embodiment of the present disclosure, a target DNA sequence methylation specific primer that could amplify a methylated CpG of the genes described herein might be used. The target DNA sequence methylation specific primer comprises at least one or more CpG dinucleotide in a region which hybridizes to the methylated CpG of the genes. Specifically, the target DNA sequence methylation specific primer for amplifying a methylated CpG of the genes comprise sequence having a homology of about 85%, about 86%, about 87%, about 88%, about 89%, about 90%, about 91%, about 92%, about 93%, about 94%, about 95%, about 96%, about 97%, about 98%, about 99%, or higher percent to sequence(s) of the primer pairs 1 to 3.

In some embodiments of the disclosure, the primer pair specific to CG2-15 is a primer pair 1 having the nucleotide sequence of cg2-15 qMSP_F (SEQ ID NO: 1; GTT AGT CGT GGG GTA AGG AGG TC) and cg2-15 qMSP_R (SEQ ID NO: 2; CCG AAA AAA ATA AAC TAA ACC CGA A); the primer pair specific to LINC00682 is a primer pair 2 having the nucleotide sequence of LI-82_qMSP_F (SEQ ID NO: 3; AGT TTA GGA TCG GTA GGG TTT TCG T) and LI-82_qMSP_R (SEQ ID NO: 4; ATT CGA CAC CTA TCC AAC TAC TCC G); the primer pair specific to NBPF13P is a primer pair 3 having the nucleotide sequence of NB-13P_qMSP_F (SEQ ID NO: 5; AGC GTT TAT AGA GTG TGG AAT CGT C) and NB-13P_qMSP_R (SEQ ID NO: 6; TTT CTC CTA CTA ATA TCT ACC CGC C).

In some embodiments of the disclosure, the probe used to detect the amplicon amplified by the primer pairs specific to CG2-15 has the sequence of SEQ ID NO: 7 (CGGTTTCGGGGAGACGGAAGCGTTTA), the probe used to detect the amplicon amplified by the primer pairs specific to LINC00682 has the sequence of SEQ ID NO: 8 (TCGTTTCGTAAATTTGTCGTTTAGTT), and the probe used to detect the amplicon amplified by the primer pairs specific to NBPF13P has the sequence of SEQ ID NO: 9 (TAGTCGTAGTTTTCGTTCGT).

In one embodiment, the detection of the methylation level of the target DNA sequence comprises the presence of hypermethylation in the target DNA sequence relative to a normal state of the target genes.

In some embodiments, the biological sample is a tissue, cell, blood, urine, serum, plasma, stool, ascites, sputum, saliva, gastric juice, bile, or oral mucosa from a human subject suspected of having lung cancer or a human subject to be detected.

As used herein, the term “who is in a need of detection of cancer” refers to an individual who has received an initial diagnosis (e.g., a CT scan showing a mass or increased biomarker level) but for whom the stage of cancer or presence or absence of methylated genes indicative of cancer is not known. The term further includes people who once had cancer (e.g., an individual in remission).

In some embodiments, a detection test to correctly predict level is measured as the sensitivity of the assay, the specificity of the assay or the area under a receiver operated characteristic (ROC) curve (AUC). The greater the area under the ROC curve, for example, the more accurate or powerful the predictive value of the test.

In one embodiment, a weighted sum score is measured to measure the methylation level in the nucleic acid sequence and genes as an indicator. The weighted sum model (WSM) is the best known and simplest multi-criteria decision analysis (MCDA)/multi-criteria decision making method for evaluating a number of alternatives in terms of a number of decision criteria.

In some embodiments, the hypermethylation status of the epigenetic biomarkers in DNA sequences described herein correlates with a “poor” prognosis or the likelihood that a subject will likely respond unfavorably to a drug or set of drugs, leading to a progression of a cancer and/or to refractory of one or more therapeutic agents. In some instances, a “poor” prognosis refers to the likelihood that a subject will not respond to a drug or set of drugs, leading to a progression of a cancer. In some instances, a “poor” prognosis refers to the survival of a subject of from less than 5 years to less than 1 month. In some instances, a “poor” prognosis refers to the survival of a subject in which the survival of the subject upon treatment is from less than 5 years to less than 1 month. In some instances, a “poor” prognosis further refers to the likelihood that a subject will develop a refractory cancer toward one or more drugs.

In some embodiments, the present disclosure provides an isolated nucleic acid molecule having a sequence selected from the group consisting of the primer pairs 1 to 3.

In some embodiments, the present disclosure provides a kit for measuring a methylation level in a human subject who is in a need of detecting a predisposition to lung cancer or predicting likelihood, treatment response, prognosis or recurrence of lung cancer in a human subject, which comprises the isolated nucleic acid molecule as described herein for assaying a methylation level of a target DNA sequence.

In some instances, the kit comprises a plurality of the target DNA sequence methylation specific primers or target DNA sequence methylation specific probes to detect or measure the methylation level/levels of one or more target DNA sequence. Such kits comprise, in some instances, at least one polynucleotide that hybridizes to at least one of the methylation biomarker sequences described herein and at least one reagent for detection of gene methylation. Reagents for detection of methylation include, e.g., sodium bisulfate, polynucleotides designed to hybridize to sequence that is the product of a marker sequence if the marker sequence is not methylated (e.g., containing at least one C-U conversion), and/or a methylation-sensitive or methylation-dependent restriction enzyme. In some cases, the kits provide solid supports in the form of an assay apparatus that is adapted to use in the assay. In some instances, the kits further comprise detectable labels, optionally linked to a polynucleotide, e.g., a probe, in the kit. In some embodiments, the kit further comprises a process unit to obtain a weighted sum score as described herein.

Optionally, one or more detectably-labeled polypeptides capable of hybridizing to the amplified portion are also included in the kit. In some embodiments, the kits comprise sufficient primers to amplify the target DNA sequences described herein, and optionally include detectably-labeled polynucleotides capable of hybridizing to each amplified DNA region or portion thereof. The kits further can comprise a methylation-dependent or methylation sensitive restriction enzyme and/or sodium bisulfite.

In some embodiments, the kits comprise sodium bisulfite, primers and adapters for whole target genes amplification, and polynucleotides (e.g., detectably-labeled polynucleotides) to quantify the presence of the converted methylated and or the converted unmethylated sequence of at least one cytosine from a DNA region of an epigenetic biomarker described herein.

In some embodiments, the kits comprise methylation sensing restriction enzymes, primers and adapters for whole target genes amplification, and polynucleotides to quantify the number of copies of at least a portion of a DNA region of an epigenetic marker described herein. In some embodiments, the kits comprise a methylation binding moiety and one or more polynucleotides to quantify the number of copies of at least a portion of a DNA region of a marker described herein.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognize that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

Although the present disclosure has been described with an exemplary embodiment, various changes and modifications may be suggested to one skilled in the art. It is intended that the present disclosure encompass such changes and modifications as fall within the scope of the appended claims.

### EXAMPLE

### Sample Preparation

Blood samples or tumor samples were collected using an ETDA-K2 tube and PAXgene Blood ccfDNA (circulating cell-free DNA) tube (QIAGEN®, Hilden, Germany, 768165) and streck cell-Free DNA BCT blood collection tubes BCT® (cfDNA; STRECK LLC, La Vista, NE U.S.A.) designed specifically for in vitro diagnostic ccfDNA testing. The samples collected using ETDA-K2 tube (BD®, Plymouth, UK, 367525) were immediately centrifuged at 2000×g for 10 min at 4° C. Within 2 h, the supernatant from each sample was transferred to a new centrifuge tube and centrifuged at 6000×g for 30 min at 4° C. and subsequently stored at −80° C. Samples collected using the PAXgene™ Blood ccfDNA tube were kept at room temperature (15-25° C.) until use within 3 days, and subsequently centrifuged at 2000×g for 10 min at 4° C. followed by 6000×g for 30 min at 4° C. for plasma separation. The plasma of each sample was split into 1.6 mL aliquots and immediately frozen at −80° C. until further use.

### The Cancer Genome Atlas Portal

The data of the Western cohort are based on data generated by The Cancer Genome Atlas (TCGA) Research Network from Genomic Data Commons (GDC) data portal. The Cancer Genome Atlas (TCGA) is a collaboration between the National Cancer Institute (NCI) and the National Human Genome Research Institute (NHGRI) that has generated comprehensive, multidimensional maps of the key genomic changes in 33 types of cancer. The TCGA dataset, comprising more than two petabytes of genomic data, is now accessible to the cancer research community to improve the prevention, diagnosis and treatment of cancer.

### Genomic DNA Extraction

Genomic DNA from matched pairs of primary tumors and adjacent lung tissues from the same patient was extracted using the QIAamp™ DNA Mini Kit (QIAGEN®, Bonn, Germany, Cat. No. 51306) according to manufacturer's instruction. After DNA quantification, the purity was verified by measuring the A260/A280 ratio (range 1.8 to 2.0) using a NanoDrop™ ND-1000 spectrophotometer (NanoDrop® Technologies Inc, Wilmington, DE, USA).

### Manual Circulating Cell-Free DNA Extraction

Circulating cell-free DNA (cfDNA) from plasma samples was extracted using the MagMAX Cell-Free DNA Isolation Kit according to the manufacturer's recommended protocol (Thermo Fisher Scientific®, Austin, TX, USA). The ccfDNA samples had clear fragment size peaks between 140 and 200 bp. The MagMAX Cell-free DNA Isolation kit provided the highest yield and low molecular weight fractions. The plasma was isolated immediately from 10 mL of peripheral blood within 2 h. After DNA quantification, the purity was verified by measuring the A260/A280 ratio (range 1.8 to 2.0) using a NanoDrop™ ND-1000 spectrophotometer (NanoDrop® Technologies, Inc., Wilmington, DE, USA).

### Automatic Circulating Cell-Free DNA Extraction and Bisulfite Conversion by KingFisher™ Duo Prime

An automated process for ccfDNA extraction and bisulfite conversion on the KingFisher™ Duo Prime purification system (ThermoFisher Scientific®, Singapore) was applied according to the manufacturer's instructions. This process fully automates magnetic bead-based DNA extraction of up to six samples simultaneously. The workflow was adapted as described in the instruction manual supplied with the MagMAX™ cell-free DNA isolation kit (ThermoFisher Scientific®, Austin, TX, USA, A29319). The ccfDNA was extracted from 1.6 mL of plasma and eluted in 60 μL of molecular biology-grade water (CORNING®, NY, USA, 46-000-CM). The bisulfite conversion cleanup was also performed on this machine for the semiautomatic assay. The automated protocol for the bisulfite conversion cleanup was developed with the instruction manual supplied with the EZ-96 DNA Methylation-Lightning™ MagPrep Kit (Zymo Research®, Irvine, CA, USA, D5046). The extracted ccfDNA was incubated with sodium bisulfate (6 M) and hydroquinone (10 mM) in a 60° C. incubator for 30 min following by the automated process. We used 60 μL of ccfDNA for bisulfite conversion, and the bisulfite-converted ccfDNA was eluted in 100 μL of molecular biology-grade water. The automated sample process was performed using a 24 deep-well plate (ThermoFisher Scientific®, Vantaa, Finland, 95040470). The eluted bisulfite-converted ccfDNA was immediately used for methylation-specific real-time PCR.

### Automatic Circulating Cell-Free DNA Extraction Using LabTurbo 24C

The automated ccfDNA extraction process was performed using the LabTurbo™ 24 Compact System (Taigen Bioscience® Co., Taipei, Taiwan) according to the manufacturer's instructions. The workflow followed the instruction manual supplied with the Labturbo™ Circulating DNA mini kit (Cat No. AIOLCD1600, Taigen Bioscience® Co., Taipei, Taiwan), with full automation of vacuum-based DNA extraction of up to 24 samples simultaneously. The ccfDNA was extracted from 1.6 mL of plasma and eluted in 60 μL of molecular biology-grade water (46-000-CM, Corning, NY, USA).

### MethylationEPIC BeadChip Array for Genome-Wide Methylation Analysis

The MethylationEPIC™ BeadChip (EPIC) array covers 850,000 CpG sites, including >90% of the CpGs and 99% Refseq genes from HM450 and an additional 413,743 CpGs. The EPIC array has been validated in comparison to the 450K platform for blood samples. The genome-wide methylation analysis was performed using the Infinium™ MethylationEPIC BeadChip array (ILLUMINA®, San Diego, CA, USA). Bisulfite conversion was performed for 500 ng of DNA using the EpiTect™ Fast DNA Bisulfite Kit (QIAGEN®, Bonn, Germany, Cat. No. 59826) according to the manufacturer's instructions. Methylation scores for each CpG site are represented as “beta” values ranging from 0 (unmethylated) to 1 (fully methylated) by determining the ratios of the methylated signal intensities to the sums of the methylated and unmethylated signal outputs. Infinium™ MethylationEPIC BeadChip data were analyzed using GenomeStudio™ Methylation Module version 2011.1. The Infinium™ MethylationEPIC BeadChip employs both Infinium I and Infinium II assays. The Infinium I assay design employs 2 bead types per CpG locus, 1 each for the methylated and unmethylated states. The Infinium II design uses 1 bead type, with the methylated state determined at the single base extension step after hybridization (right panel). A differentially methylated CpG heatmap of the target genes was visualized by a heatmap using heatmapper software. A gradient-scale heatmap was used to visualize the DNA methylation level from low to high.

Probe-Based Quantitative Methylation-Specific PCR (qMSP)

After bisulfite conversion of DNA, which was done according to the manufacturer's recommended protocol, the DNA methylation levels of CG-15, LINC00682 and NBPF13P were measured using TaqMan™ quantitative methylation-specific PCR (qMSP) with a LightCycler 96 (Roche Applied Science®, Penzberg, Germany). qMSP was performed using the SensiFAST™ Probe No-ROX Kit (Bioline®, London, UK, Cat. No. BIO-86020) with specific primers and methyl-TaqMan probes of candidate genes. Normalized DNA methylation values, which were calibrated to the control group, were obtained using LightCycler™ Relative Quantification software (Version 1.5, Roche Applied Science®). The beta-actin (ACTB) gene was used as methylation-independent DNA control. The primers/probes for the ACTB gene were designed without the CpG site (as a control for input DNA). The primers/probes for candidate genes were designed on their methylated promotor regions, especially on the identified differential regions between normal and tumor tissues. According to the sequencing results, only when all CpG sites are methylated can a successful PCR reaction occur. The target genes were considered hypermethylated when the methylation level relative to that of the ACTB gene was at least 2-fold higher in the lung tumor compared with the paired normal lung tissue sample. The specificity of the candidate gene methylation end products was confirmed by bisulfite sequencing. The primers used for qMSP are the primer pairs 1 to 3.

### Statistical Analysis

The Pearson's chi-squared test, Mann-Whitney U test, Wilcoxon test and Spearman's rank correlation analyses were performed using SPSS (IBM®, Armonk, NY, USA). The Pearson's chi-squared test was used to compare lung cancer patients in terms of candidate gene methylation, RNA expression and other clinical data. The paired-sample Wilcoxon test and t-test was used to compare differences in DNA methylation between tumors and matched adjacent normal tissues, different cancer types, as well as in candidate ccfDNA methylation between surgery treatment in lung cancer patients. The Spearman's rank correlation was adopted to analyze the methylation levels of the tumor and plasma samples.

### Example 1 Methylation Level of Target DNA Sequence in Lung Cancer Tissue

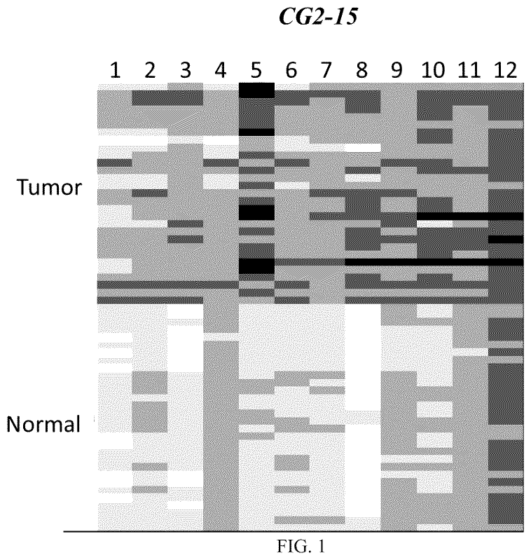

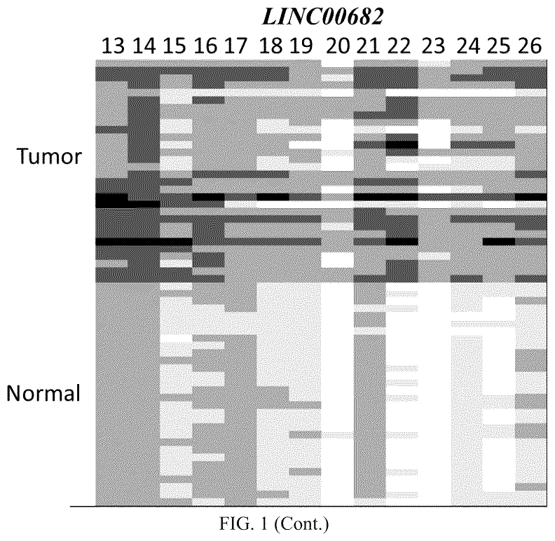

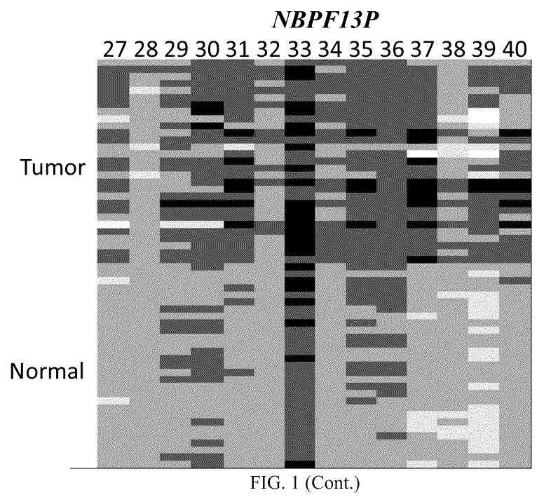

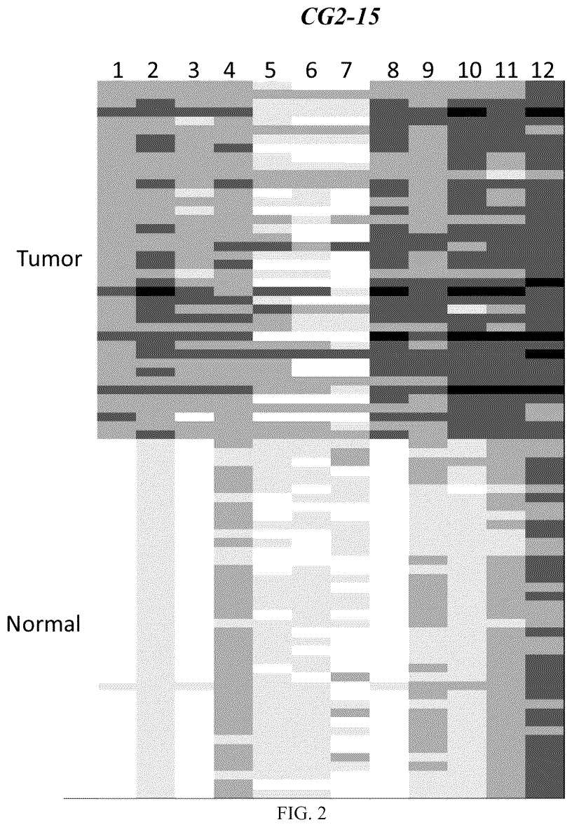

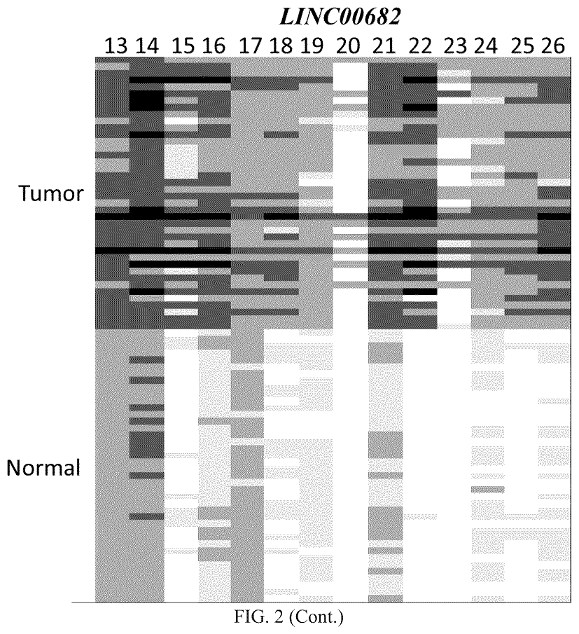

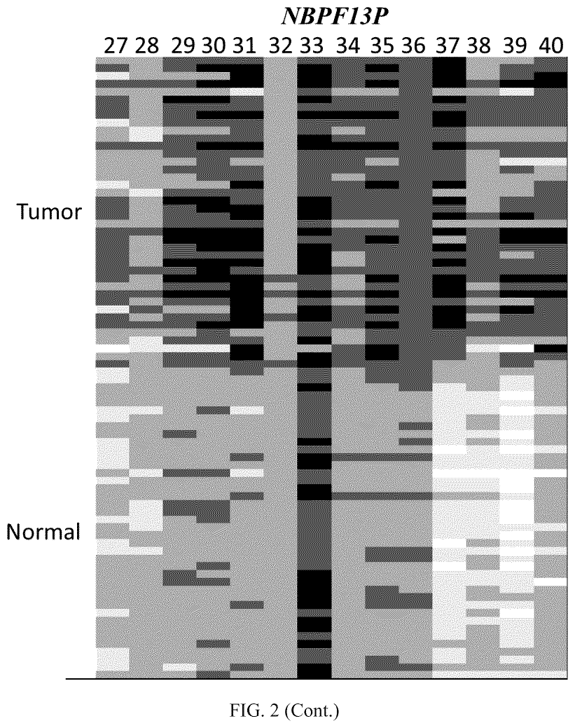

The β value for Illumina Methylation 450K array-based data was generated from The Cancer Genome Atlas (TCGA) Research Network. The target nucleic acid and genes were selected when β value from normal tissues is less than 0.15; Δβ value (the value of Tumor subtracts that of normal tissues) is higher than 0.5 or lower than 0.25. The methylation levels Δβ value (T) of the target DNA sequences are shown in Tables 1a and 1b. The difference of methylation level (β value) of target nucleic acid and genes between lung adenocarcinoma or lung squamous cell carcinoma tissues and adjacent normal tissues is shown in FIGS. 1 and 2.

TABLE la
Lung adenocarcinoma
Patients with Lung adenocarcinoma

| | β value | β value |
|---|---|---|
| Avg | (Tumor) | (Normal) |
| CG2-15 | 0.603 | 0.178 |
| LINC00682 | 0.494 | 0.083 |
| NBPF13P | 0.651 | 0.270 |

TABLE 1b
Lung squamous cell carcinoma
Patients with Lung squamous cell carcinoma

| | β value | β value |
|---|---|---|
| Avg | (Tumor) | (Normal) |
| CG2-15 | 0.574 | 0.037 |
| LINC00682 | 0.598 | 0.044 |
| NBPF13P | 0.571 | 0.126 |

β value (T), methylation level of tumor tissues; β value (T)≥0.5 will be calculated as hypermethylation biomarkers.

### Example 2 Early Detection of Methylation Level of Epigenetic Biomarkers of Target Genes in Plasma Samples of Healthy Subjects and Lung Cancer Patients

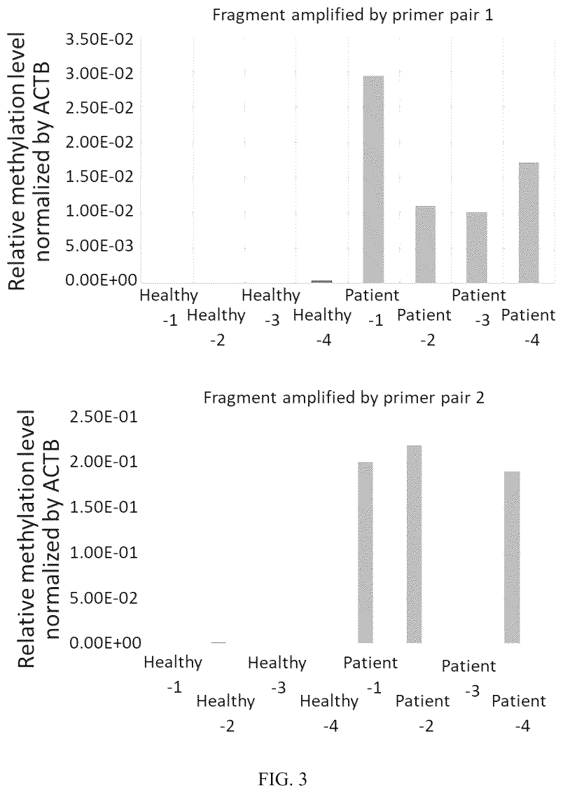

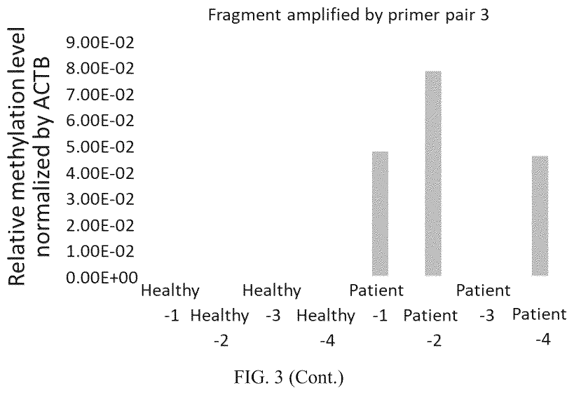

The circulating cell-free DNA was extracted from plasma. Briefly, 3.5 mL of plasma was isolated immediately from 10 mL of peripheral blood. After circulating cell-free DNA (cfDNA) was extracted from plasma that obtained from colorectal cancer patients and healthy subjects, cfDNA was performed by bisulfite conversion. Probe-based methylation specific real-time PCR (qMSP) was used for cfDNA methylation analyses. The difference in early detection of the methylation level of epigenetic biomarkers of target genes in plasma samples of healthy subjects and lung cancer patients is shown in FIG. 3.

Our findings demonstrated significantly higher methylation levels of NBPF13P and CG2-15 in the plasma of lung cancer patients compared to those of healthy individuals.

FIG. 4 shows the differences in early detection of the methylation level of target genes in plasma samples of healthy subjects and lung cancer patients in pilot study.

These results suggest that cfDNA in lung cancer patient plasma may exhibit higher methylation levels of NBPF13P and CG2-15 than in healthy individuals.

In an expanded cohort following the initial pilot study, the results demonstrated that the overall methylation levels of NBPF13P and CG2-15 in 20 lung cancer patients were distinct from those in 66 healthy individuals. Notably, CG2-15 exhibited significantly higher methylation levels even in early-stage patient plasma samples.

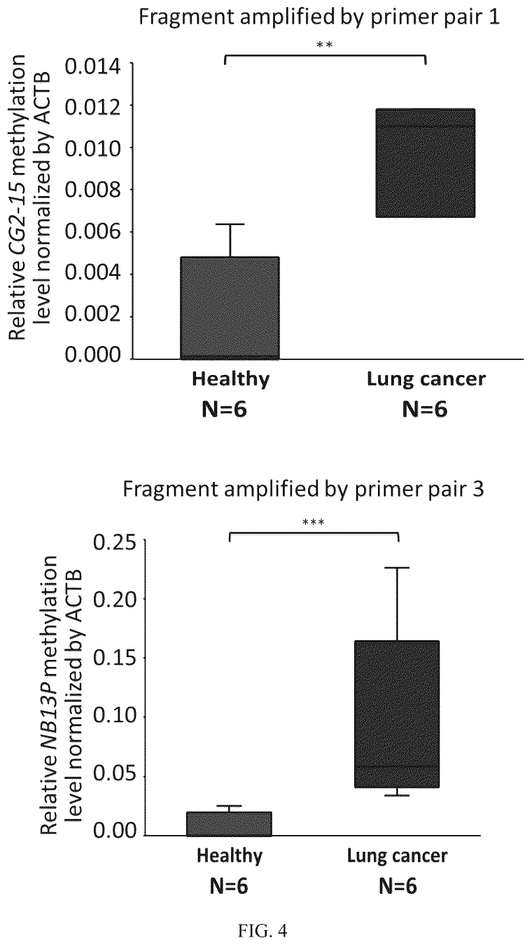

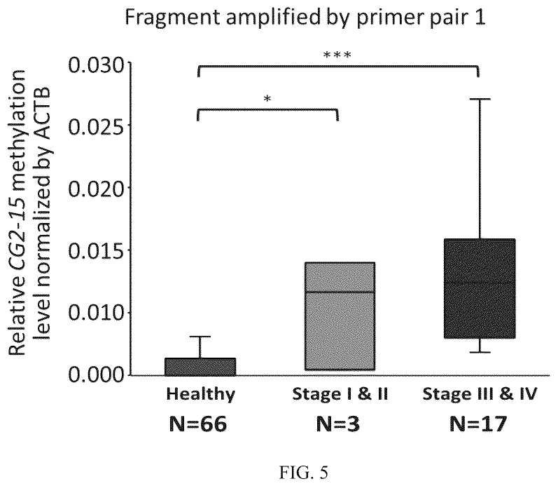

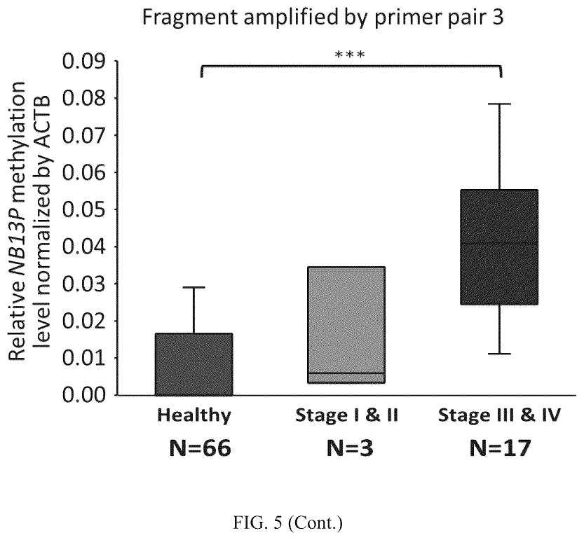

FIG. 5 shows the differences in early detection of the methylation level of target genes in plasma samples from the expanded cohort.

### Fragment Amplified by Primer Pair 1

### Example 3 Performance of the Early Detection of Methylation Levels of Target Genes in Plasma Samples of Lung Cancer Patients and Healthy Subjects

To evaluate the utility of analyzing the methylation levels of NBPF13P and CG2-15 for early detection of lung cancer, we compared the results of this analysis in 86 individuals, with or without lung cancer. Using receiver operating characteristic (ROC) curve analysis, the results revealed that the most optimal model was established based on the methylation levels of both NBPF13P and CG2-15 with an AUC value of 0.96, a sensitivity of 90.0%, specificity of 90.9% and 90.7% of accuracy.

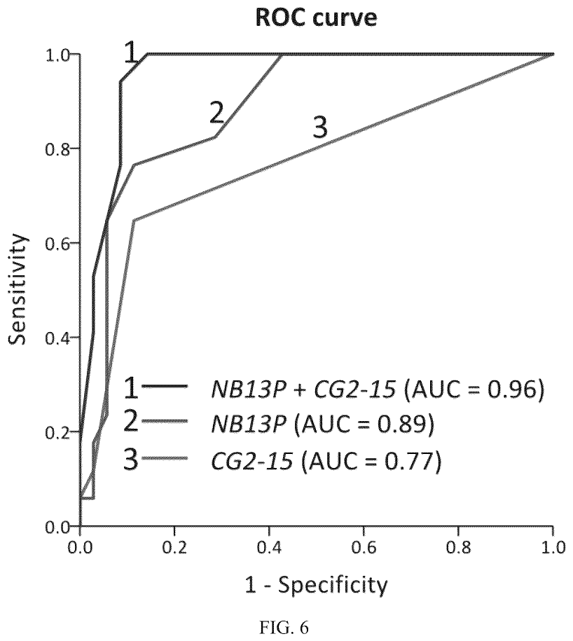

FIG. 6 and Table 2 show the clinical performance based on the methylation level of target genes in plasma samples.

TABLE 2
Detection performance of NBPF13P and CG2-15 in cfDNA as biomarkers
for early detection of lung cancer

| | Lung cancer patients | Healthy | Measures |
|---|---|---|---|
| Positive Detection | True positive | False positive | PPV ª |
| | 18 | 6 | 75.0% |
| Negative Detection | False negative | True negative | NPV ^{b} |
| | 2 | 60 | 96.8% |
| Measures | Sensitivity | Specificity | Accuracy |
| | 90.0% | 90.9% | 90.7% |

^{a} PPV = Positive predictive value
^{b} NPV = Negative predictive value

We also analyzed the results of the population who also received CEA testing. Among the 50 healthy individuals and 14 lung cancer patients with available test values, the model based on CEA levels showed an AUC of 0.85, with a sensitivity of 64.3% and a specificity of 96.7%. Conversely, the model using the methylation levels of NBPF13P and CG2-15 reached an AUC of 0.98, with a sensitivity of 100% and a specificity of 93.3%. These findings suggest that methylation levels of NBPF13P and CG2-15 offer more accurate predictions for lung cancer probability compared to the currently employed CEA biomarker.

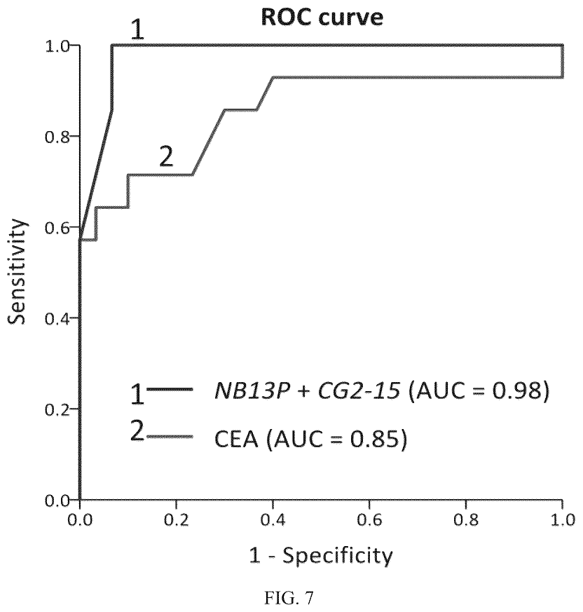

FIG. 7 shows a comparative analysis of the clinical performance between the methylation levels of NBPF13P and CG2-15 versus CEA in identifying lung cancer probability.

### Example 4 Predicting Treatment Response of Lung Cancer

The target DNA sequences and methylation level are used in predicting treatment response of lung cancer.

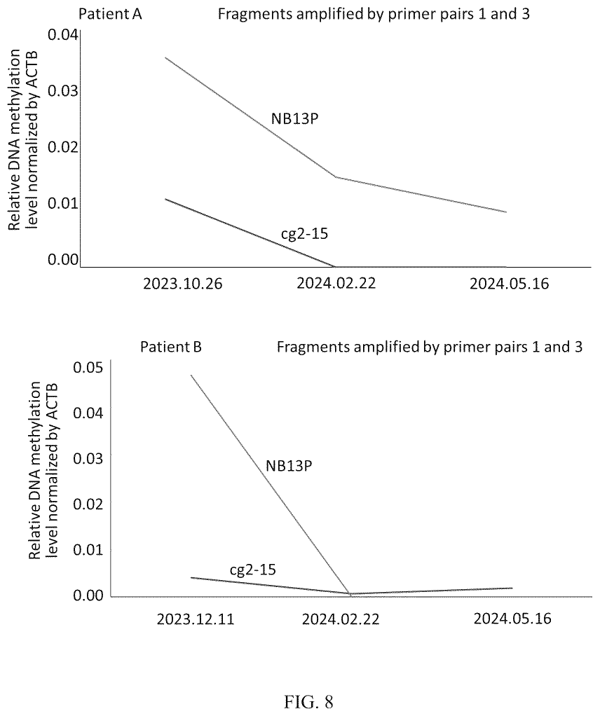

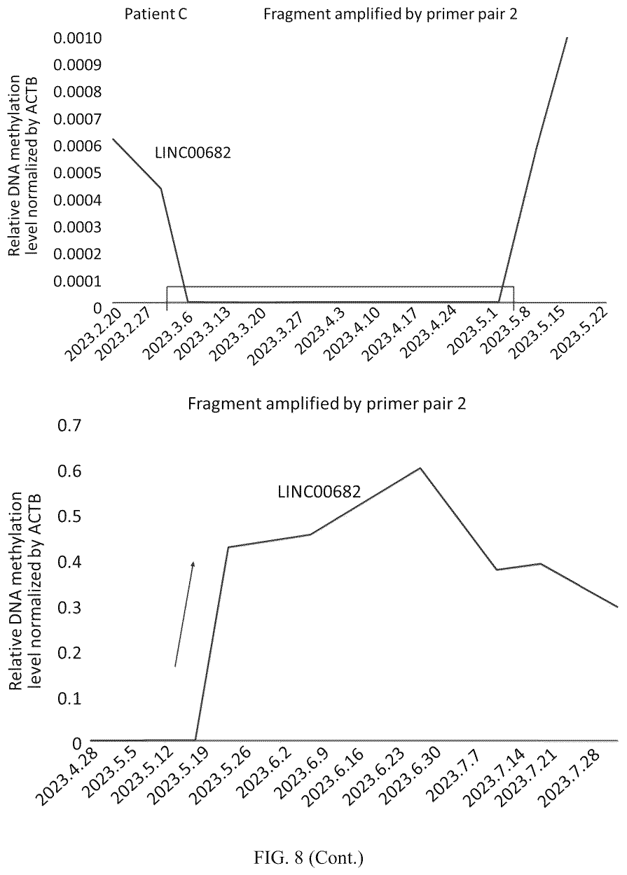

As shown in FIG. 8, Patient A showed improvement after receiving targeted therapy, with a tendency for the signal to weaken.

As shown in FIG. 8, Patient B showed improvement after receiving targeted therapy, with a tendency for the signal to weaken.

As shown in FIG. 8, Patient C received immunotherapy during the period of the red box in the chart and showed improvement in the conditions, with a tendency for the signal to weaken.

As shown in FIG. 8, when the conditions were deteriorated, a tendency for the detection signal increases.

What is claimed is:

1. A method for assaying a methylation level in a human subject who is in a need of detecting a predisposition to lung cancer or predicting likelihood, treatment response, prognosis or recurrence of the lung cancer, comprising assaying the methylation level of a target DNA sequence in one or more target genes selected from CG-15, LINC00682 and NBPF13P, wherein the presence of hypermethylation in the target DNA sequence of the subject is indicative of the predisposition to, likelihood, poor treatment response, poor prognosis or recurrence of lung cancer.

2. The method of claim 1, wherein the one or more target genes are CG2-15; LINC00682; NBPF13P; CG2-15 and LINC00682; CG2-15 and NBPF13P; LINC00682 and NBPF13P; or CG-15, LINC00682 and NBPF13P.

3. The method of claim 1, wherein the methylation level is assayed by a methylation-specific PCR (MSP), quantitative methylation-specific PCR (QMSP), methylation DNA immunoprecipitation PCR (MeDIP), bisulfite conversion, bisulfite sequencing (BS), pyrosequencing, mass spectrometry, methylation specific nuclease, mass-based separation, target capture or microarrays.

4. The method of claim 1, which further comprises steps of providing (i) a biological sample containing DNA from the human subject and extracting cell-free DNA from the biological sample or (ii) a biological sample from a lung tumor tissue of the human subject and extracting the genomic DNA from the biological sample, wherein the cell-free DNA or the genomic DNA comprises the target DNA sequence in the one or more target genes.

5. The method of claim 1, wherein the target DNA sequence in CG2-15 comprises a fragment amplified by a primer pair 1 having the nucleotide sequence of SEQ ID NOs: 1 (GTT AGT CGT GGG GTA AGG AGG TC) and 2 (CCG AAA AAA ATA AAC TAA ACC CGA A) or fragment thereof having an identity of at least 85% to the primer pair 1; the target DNA sequence in LINC00682 comprises a fragment amplified by a primer pair 2 having the nucleotide sequence of SEQ ID NOs: 3 (AGT TTA GGA TCG GTA GGG TTT TCG T) and 4 (ATT CGA CAC CTA TCC AAC TAC TCC G) or a fragment thereof having an identity of at least 85% to the primer pair 2, and/or the target DNA sequence in NBPF13P comprises a fragment amplified by a primer pair 3 having the nucleotide sequence of SEQ ID NOs: 5 (AGC GTT TAT AGA GTG TGG AAT CGT C) and 6 (TTT CTC CTA CTA ATA TCT ACC CGC C) or a fragment thereof having an identity of at least 85% to the primer pair 3.

6. The method of claim 5, wherein the target DNA sequence comprises any of the following combinations:
the fragment amplified by the primer pair 1 or the fragment thereof and the fragment amplified by the primer pair 2 or the fragment thereof,
the fragment amplified by the primer pair 1 or the fragment thereof and the fragment amplified by the primer pair 3 or the fragment thereof,
the fragment amplified by the primer pair 2 or the fragment thereof and the fragment amplified by the primer pair 3 or the fragment thereof, and
the fragment amplified by the primer pair 1 or the fragment thereof, the fragment amplified by the primer pair 2 or the fragment thereof and the fragment amplified by the primer pair 3 or the fragment thereof.

7. The method of claim 1, wherein assaying the methylation level further comprises a step of measuring the specificity and sensitivity by a weighted sum score analysis.

8. The method of claim 1, which comprises
(a) providing a biological sample from the human subject containing a target DNA sequence in one or more target genes selected from CG-15, LINC00682 and NBPF13P, and
(b) assaying the methylation level of the target DNA sequence in CG2-15, wherein the target DNA sequence comprises a fragment amplified by a primer pair 1 having the nucleotide sequence of SEQ ID NOs: 1 and 2 or fragment thereof having an identity of at least 85% to the primer pair 1;
assaying the methylation level of the target DNA sequence in LINC00682, wherein the target DNA sequence comprises a fragment amplified by a primer pair 2 having the nucleotide sequence of SEQ ID NOs: 3 and 4 or a fragment thereof having an identity of at least 85% to the primer pair 2, and/or
assaying the methylation level of the target DNA sequence in NBPF13P, wherein the target DNA sequence comprises a fragment amplified by a primer pair 3 having the nucleotide sequence of SEQ ID NOs: 5 and 6 or a fragment thereof having an identity of at least 85% to the primer pair 3,
wherein the presence of hypermethylation in the target DNA sequence of the human subject is indicative of the predisposition to, likelihood, poor treatment response, poor prognosis or recurrence of lung cancer.

9. The method of claim 8, wherein the biological sample providing step comprises providing (i) a biological sample containing DNA from the human subject and extracting cell-free DNA from the biological sample or (ii) a biological sample from a lung tumor tissue of the human subject and extracting the genomic DNA from the biological sample, wherein the cell-free DNA or the genomic DNA comprises the target DNA sequence in the one or more target genes; and
wherein before the assaying step, which further comprises treating the extracted cell-free DNA or the extracted genomic DNA with bisulfate;
amplifying the bisulfite-treated DNA with at least one of primer pairs 1 to 3 or a fragment thereof; and
measuring the methylation level of one or more CpG sites in target DNA sequence by a polymerase chain reaction.

10. The method of claim 8, wherein the methylation level is measured by a methylation-specific PCR (MSP), quantitative methylation-specific PCR (QMSP), methylation DNA immunoprecipitation PCR (MeDIP), bisulfite conversion, bisulfite sequencing (BS), pyrosequencing, mass spectrometry, methylation specific nuclease, mass-based separation, target capture or microarrays.

11. The method of claim 8, wherein the target DNA sequence comprises any of the following combinations:
the fragment amplified by the primer pair 1 or the fragment thereof and the fragment amplified by the primer pair 2 or the fragment thereof,
the fragment amplified by the primer pair 1 or the fragment thereof and the fragment amplified by the primer pair 3 or the fragment thereof,
the fragment amplified by the primer pair 2 or the fragment thereof and the fragment amplified by the primer pair 3 or the fragment thereof, and
the fragment amplified by the primer pair 1 or the fragment thereof, the fragment amplified by the primer pair 2 or the fragment thereof and the fragment amplified by the primer pair 3 or the fragment thereof.

12. The method of claim 1, wherein the lung cancer is squamous cell carcinoma or adenocarcinoma.

13. The method of claim 4, wherein the biological sample is a tissue, cell, blood, urine, serum, plasma, stool, ascites, sputum, saliva, gastric juice, bile, or oral mucosa.

14. The method of claim 8, wherein the biological sample is a tissue, cell, blood, urine, serum, plasma, stool, ascites, sputum, saliva, gastric juice, bile, or oral mucosa.

15. A method for assaying a methylation level in a biological sample from a human subject who is in a need of detecting a predisposition to lung cancer or predicting likelihood, treatment response, prognosis or recurrence of the lung cancer, comprising
providing a biological sample containing a target DNA sequence in one or more target genes selected from CG-15, LINC00682 and NBPF13P from the human subject;
assaying methylation levels of the CG-15, LINC00682 and NBPF13P from the biological sample using methylation specific polymerase chain reaction assay (MSP), wherein probes used in the assay comprises SEQ ID NO: 7 for CG-15, SEQ ID NO: 8 from LINC00682 and SEQ ID NO: 9 for NBPF13P;
comparing the methylation levels of the previous step to methylation levels from a control sample without lung cancer; and
identifying the human subject as having the predisposition to, likelihood, poor treatment response, poor prognosis or recurrence of, lung cancer when hypermethylation is present in the relative methylation level.

16. The method of claim 15, wherein assaying the methylation level further comprises a step of measuring the specificity and sensitivity by a weighted sum score analysis.

17. The method of claim 15, wherein the lung cancer is squamous cell carcinoma or adenocarcinoma.

18. The method of claim 15, wherein the biological sample is a tissue, cell, blood, urine, serum, plasma, stool, ascites, sputum, saliva, gastric juice, bile, or oral mucosa.

19. An isolated nucleic acid molecule having a sequence selected from the group consisting of:
a primer pair 1 having the nucleotide sequence of SEQ ID NOs: 1 and 2 or a fragment thereof having an identity of at least 85%,
a primer pair 2 having the nucleotide sequence of SEQ ID NOs: 3 and 4 or a fragment thereof having an identity of at least 85%, and
a primer pair 3 having the nucleotide sequence of SEQ ID NOs: 5 and 6 or a fragment thereof having an identity of at least 85%.

20. A kit for detecting a predisposition to lung cancer or predicting likelihood, treatment response, prognosis or recurrence of lung cancer in a subject, which comprises the isolated nucleic acid molecule of claim 19.


SEQUENCE LISTING (9 sequences)

SEQ ID NO: 1 - DNA, 23 bp, synthetic construct
  source 1..23: mol_type = other DNA
gttagtcgtggggtaaggaggtc

SEQ ID NO: 2 - DNA, 25 bp, synthetic construct
  source 1..25: mol_type = other DNA
ccgaaaaaaataaactaaacccgaa

SEQ ID NO: 3 - DNA, 25 bp, synthetic construct
  source 1..25: mol_type = other DNA
agtttaggatcggtagggttttcgt

SEQ ID NO: 4 - DNA, 25 bp, synthetic construct
  source 1..25: mol_type = other DNA
attcgacacctatccaactactccg

SEQ ID NO: 5 - DNA, 25 bp, synthetic construct
  source 1..25: mol_type = other DNA
agcgtttatagagtgtggaatcgtc

SEQ ID NO: 6 - DNA, 25 bp, synthetic construct
  source 1..25: mol_type = other DNA
tttctcctactaatatctacccgcc

SEQ ID NO: 7 - DNA, 26 bp, synthetic construct
  source 1..26: mol_type = other DNA
cggtttcggggagacggaagcgttta

SEQ ID NO: 8 - DNA, 26 bp, synthetic construct
  source 1..26: mol_type = other DNA
tcgtttcgtaaatttgtcgtttagtt

SEQ ID NO: 9 - DNA, 20 bp, synthetic construct
  source 1..20: mol_type = other DNA
tagtcgtagttttcgttcgt